(19) United States
(12) Patent Application Publication
(10) Pub. No.: US 2026/0317042 A1
(43) Pub. Date: Oct. 15, 2026

(54) AEROSOL PRODUCTS

(71) Applicant: THE PROCTER & GAMBLE COMPANY, Cincinnati, OH (US)

(72) Inventors: Julie SAVCHENKO, Maineville, OH (US); Shawn David MCCONAUGHY, Cincinnati, OH (US); Matthew John MARTIN, California, KY (US); David Frederick Swaile, Cincinnati, OH (US)

(21) Appl. No.: 19/648,191

(22) Filed: Apr. 15, 2026

Related U.S. Application Data
(60) Provisional application No. 63/789,034, filed on Apr. 15, 2025.

(51) Int. Cl. B65D 83/141; B65D 83/14

(52) U.S. Cl. CPC B65D 83/141; B65D 83/754

(57) ABSTRACT

Aerosol products utilizing a combination of a non-hydrocarbon propellant (e.g., compressed gas) and adsorbent material to provide a consistent experience throughout the life of the product. A filter is employed to help mitigate escape of adsorbent material from a container storing the same. The filter is configured and positioned so as not to interfere with a product composition reservoir as it is filled during manufacture of the aerosol products. The aerosol products are ergonomically designed to enable users to easily hold and manipulate the container to precisely apply a product composition to their desired areas, such as underarms, feet, hair, and intimate areas.

### FIELD

The present disclosure generally relates to aerosol products that utilize a non-hydrocarbon propellant, particularly a compressed gas. The aerosol products can be useful for dispensing product compositions related to personal care, fabric care, air care, hard and soft surface care, and the like.

### BACKGROUND

Some consumers prefer aerosol products, including personal care products such as hairsprays, deodorants, and antiperspirants. These products are released as a fine mist that allows for broad coverage, making it easy to apply the product to a larger area or hard-to-reach places. Additionally, the mist dries quickly upon application, providing a non-greasy and non-sticky feel.

Many aerosol products use a hydrocarbon propellant. However, some consumers would prefer a product that utilizes a non-hydrocarbon propellant, such as compressed gases, which are in the gas state at 25° C. and at a pressure of at least 50 psi, such as air, nitrogen, oxygen, carbon dioxide, or mixtures thereof. Creating a consumer-acceptable aerosol product that utilizes a compressed gas propellant can however be challenging. Unlike hydrocarbon propellants, compressed gases are always in a vapor state. As a result, the pressure inside the container decreases as the product is dispensed, posing difficulties in maintaining an aerosol droplet size distribution that is acceptable. This is particularly crucial for personal care compositions, as the product's effectiveness can be compromised if the droplet size falls outside a range preferred by consumers.

U.S. Pat. No. 12,023,703 (hereinafter “'703 Patent”) discloses one solution that employs bag-on-valve aerosol technology and the inclusion of adsorbent material in combination with the compressed gas propellant to help maintain a consistent consumer experience throughout the life of the product. FIGS. 1 and 2 show a representation of the teachings of the '703 Patent. The figures show a dispenser 10 that includes a container 12, adsorbent material 14, a valve assembly 16, and a mounting cup 18 for both sealing the container and for housing valve assembly 16. A first dip tube 20 and second dip tube 22 extend from valve assembly 16. A distal portion of first dip tube 20 is positioned within a product reservoir 30. Second dip tube 22 contains a filter or frit 40 to prevent adsorbent material 14 from passing into the valve and being ultimately dispensed along with the consumer product.

A downside associated with the design taught in the '703 Patent is that second dip tube 22 is located next to product reservoir 30. The location and long extension of second dip tube 22 can interfere with product reservoir 30 as product is charged into product reservoir 30. FIG. 2 illustrates the interference can be so extreme that second dip tube 22 is displaced as product reservoir 30 expands upon filling. Second dip tube 22 along with frit 40 can become damaged, such as being cracked, ruptured or detached from valve assembly 16 whereby its filtering function is compromised. The interaction between second dip tube 22 and product reservoir 30 can also prevent the product reservoir from completely expanding thereby reducing the amount it can receive during the charging process. This interaction can even damage product reservoir 30, which could lead to product escaping the reservoir and reacting with the propellant and/or adsorbent material 14 to cause an increase in pressure within container 12, potentially causing it to rupture or explode.

Thus, there is a continued need for improved design of aerosolized products that utilize a non-hydrocarbon propellant, such as compressed gas.

### SUMMARY

Improved aerosol products are provided herein. The aerosol products are functionally designed to maintain a consistent consumer experience over the life of the product. The products are also ergonomically designed to enable users to easily hold the product container and precisely apply product compositions to their desired areas, including, for example, underarms, feet, hands, chest (including under one's boobs), stomach, legs, and intimate areas.

### BRIEF DESCRIPTION OF THE DRAWINGS

The above-mentioned and other features and advantages of the present disclosure, and the manner of attaining them, will become more apparent and the disclosure itself will be better understood by reference to the following description of example forms of the disclosure taken in conjunction with the accompanying drawings, wherein:

FIG. 1 is a side view of a prior art aerosol product before its product reservoir is filled;

FIG. 2 is a side view of the prior art aerosol product shown in FIG. 1 after its product reservoir is at least partially filled;

FIG. 3 is a side view of an exemplary aerosol product provided herein before its product reservoir is filled;

FIG. 4 is a side view of the aerosol product shown in FIG. 3 after its product reservoir is at least partially filled;

FIG. 5 is a partial side view of another exemplary aerosol product provided herein, showing a first alternative filter configuration and position;

FIG. 6 is a partial side view of another exemplary aerosol product provided herein, showing a second alternative filter configuration and position;

FIGS. 7-10 are cross-sectional views of another exemplary aerosol product provided herein, showing disposition of a filter within the actuator;

FIG. 11 is a partial side view of another exemplary aerosol product provided herein, showing an alternative filter configuration and position;

FIG. 12 is a partial side view of another exemplary aerosol product provided herein, showing an alternative filter configuration and position; and

FIG. 13 is a perspective view of an exemplary product reservoir provided herein.

### DETAILED DESCRIPTION

Reference within the specification to “form(s)”, “aspect(s)”, “embodiment(s)” or the like means that a particular material, feature, structure and/or characteristic described in connection with the form/aspect/embodiment is included in at least one form/aspect/embodiment, optionally several forms/aspects/embodiments, but it does not mean that all forms/aspects/embodiments incorporate the material, feature, structure, and/or characteristic described. Furthermore, materials, features, structures and/or characteristics may be combined in any suitable manner across different forms/aspects/embodiments, and materials, features, structures and/or characteristics may be omitted or substituted from what is described. Thus, forms/aspects/embodiments described herein may comprise or be combinable with elements or components of other forms/aspects/embodiments despite not being expressly exemplified in combination, unless otherwise stated or an incompatibility is stated.

All numeric ranges are inclusive and combinable. For example, the numeric ranges are inclusive of narrower ranges, and delineated upper and lower range limits are interchangeable to create further ranges not explicitly delineated.

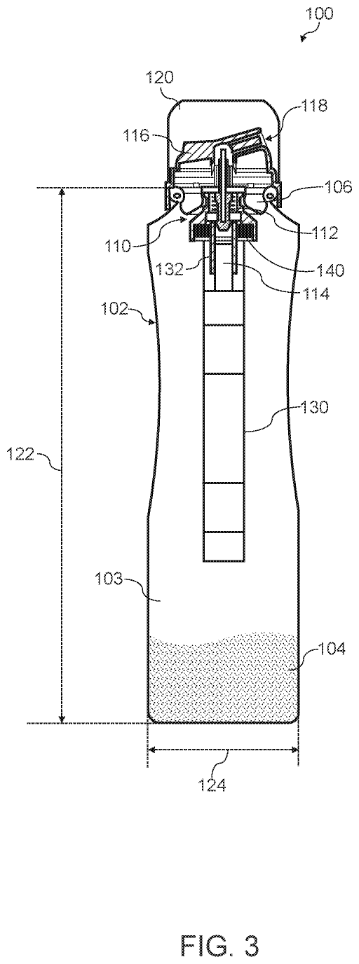

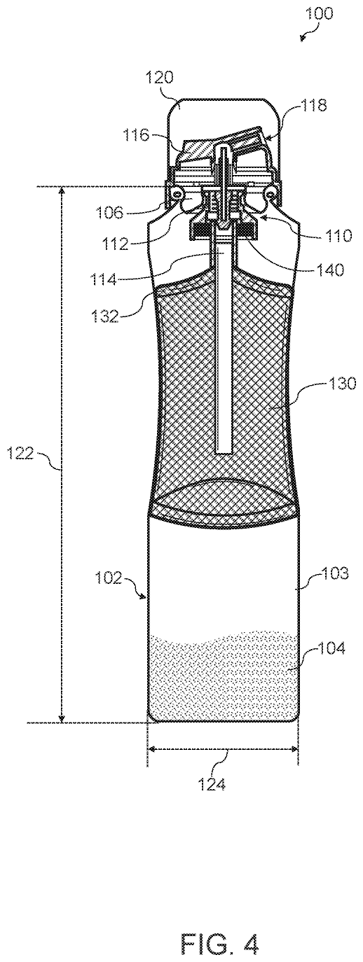

Improved aerosol products are provided. FIGS. 3 and 4 show an exemplary aerosol product 100 comprising a container 102, adsorbent material 104 (e.g., activated carbon, zeolites, metal-organic frameworks, porous silica, amine-functionalized materials, biochar, graphene-based materials, or combinations thereof) disposed in a lower portion of container cavity 103, dispensing assembly 110, and product reservoir 130. Dispending assembly 110 is in sealing engagement with container 102 via a top member 106 in the form of a mounting cup. Dispensing assembly 110 includes a valve 112, a conduit 114 in the form of a dip tube for conveying product composition from product reservoir 130 to valve 112, an actuator 116 for operating valve 112, and a spray nozzle 118. Actuator 116 can be a locking actuator or a non-locking actuator. A locking actuator can include a twist-to-lock mechanism, for example, to prevent accidental actuation. A protective cap 120 is removably installed onto actuator 116 to prevent unintended product dispensing.

A non-hydrocarbon propellant (e.g., compressed gas including compressed air, nitrogen, oxygen, carbon dioxide, or mixtures thereof) can be added to cavity 103 via valve 112. On actuation, the propellant is released together with a portion of a product composition in product reservoir 130. The propellant and product composition mix as they pass through valve 112 or actuator 116 to exit container 102 via spray nozzle 118.

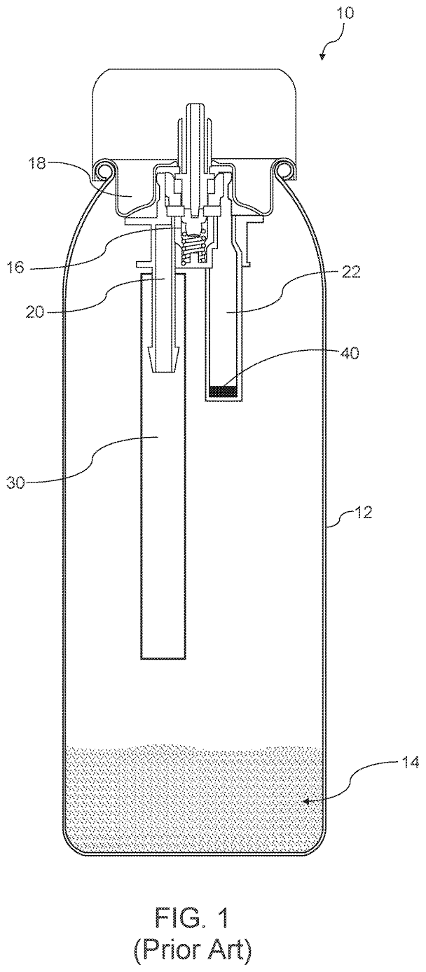

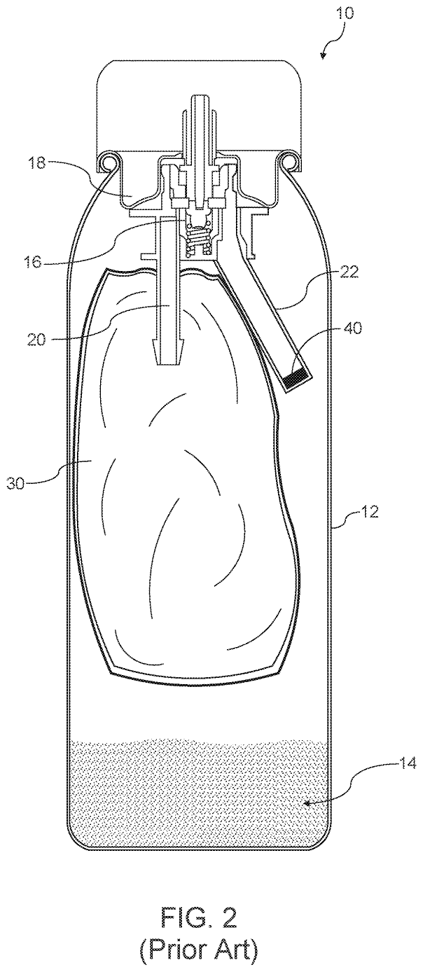

As noted in the Background Section, creating a consumer-acceptable aerosol product that utilizes a non-hydrocarbon propellant can be challenging. Unlike hydrocarbon propellants, compressed gases are always in a vapor state. As a result, the pressure inside the container decreases as the aerosolized product is dispensed, which can prevent the complete emptying of the product reservoir and/or yield inconsistent spray properties including droplet size and spray rate. Inclusion of adsorbent material in an aerosol cannister has been found to improve the overall experience of aerosol products that utilize a non-hydrocarbon propellant. A filter can be employed for mitigating escape of adsorbent material from the container. Adding an additional structural element, such as a filter or frit, into an already crowded aerosol container can be challenging and result in compromises to product functionality. As shown in FIGS. 1 and 2, a prior art aerosol product 10 includes a filter assembly comprising a tube 22 and frit or filter 40, which extends along a significant length of its product reservoir 30. The configuration and positioning of the filter creates an interference with the product reservoir during filling, which can lead to several different problems. The inventors have discovered filter configurations and positioning to improve upon the prior art approach.

Referring again to FIGS. 3 and 4, exemplary product 100 includes a filter 140 positioned in an area of cavity 103 that is between a top sealed portion 132 of product reservoir 130 and mounting cup 106. With this improved design approach, and as shown in FIG. 4, product reservoir 130 can be filled completely without interference from filter 140.

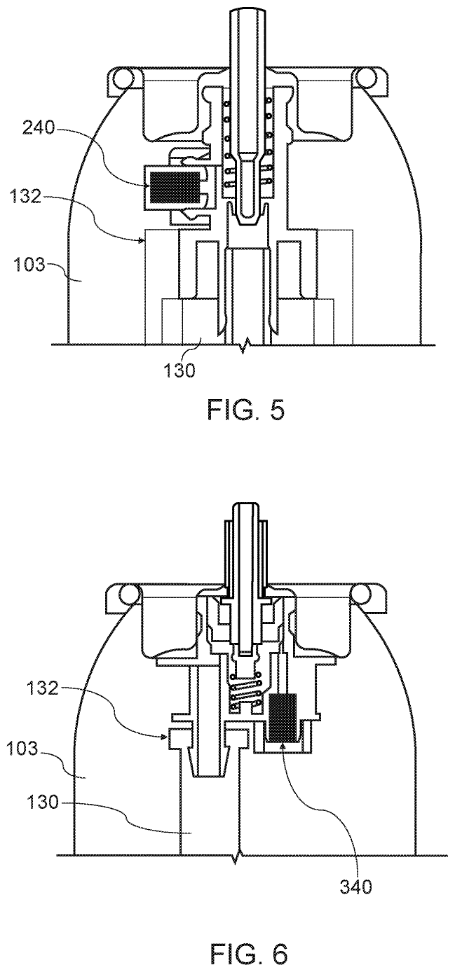

FIGS. 5 and 6 respectively illustrate alternative filter 240/340 configurations and positioning, wherein the filter is housed within container cavity 103 but generally located proximate to or above a top sealed portion 132 of product reservoir 130.

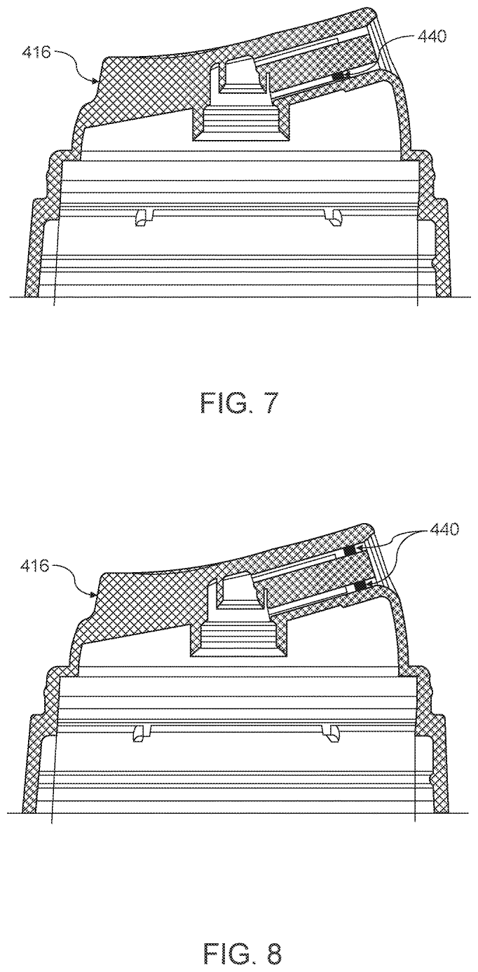

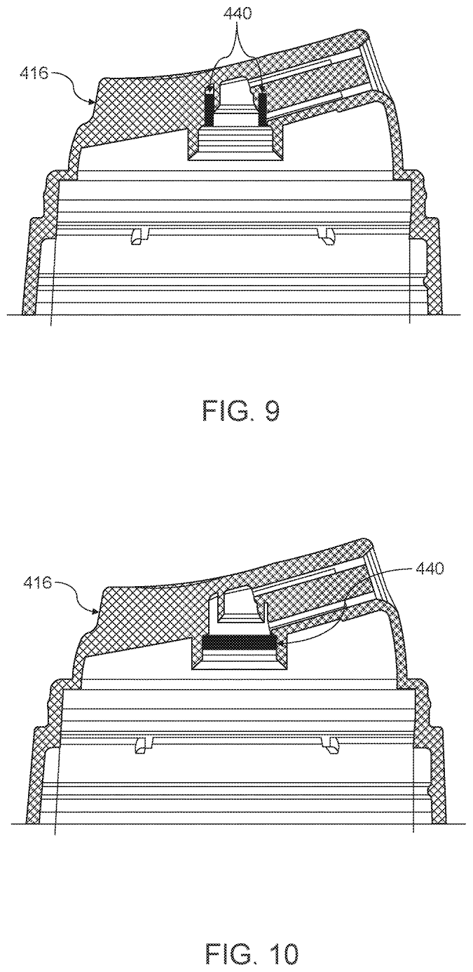

A non-interfering filter position can also occur between the valve and actuator, or within the actuator itself. FIGS. 7-10 are cross-sectional views to illustrate examples of a filter 440 disposed within the actuator 416 itself.

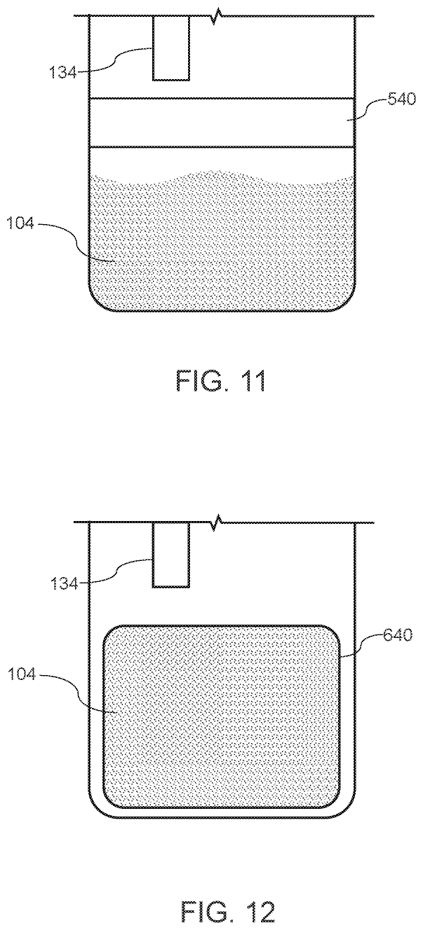

Referring now to FIGS. 11 and 12, a filter 540 can be employed below a bottom sealed portion 134 of product reservoir 130 and above adsorbent material 104, as shown in FIG. 11. In FIG. 12, filter embodiment 640 is a porous container that encloses adsorbent material 104. Filters 540/640 can be rigid or flexible. In one form, filter 640 is a porous sachet.

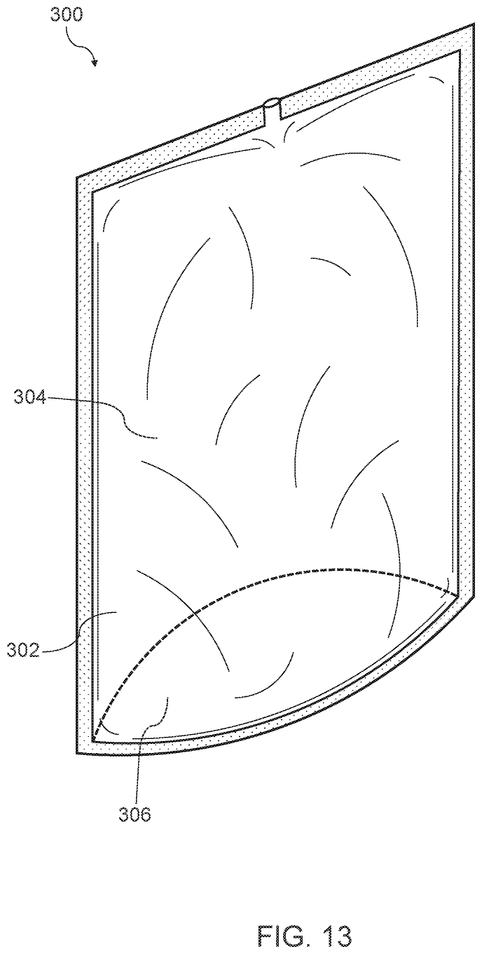

The product reservoir can have a variety of different configurations. The product reservoir configuration can decrease the amount of residual product composition left in the reservoir (e.g., less than 7, 5, or 3 milliliters) at the end of the aerosol product's life. In one form, the product reservoir is a simple pouch constructed from two sheets sealed to one another so that the reservoir essentially only comprises two opposing panels which can improve the capacity of the product reservoir and/or increase the efficiency of reservoir expansion upon filling. While this design can be employed in the aerosol products provided herein, an alternative design incorporates bags or pouches that have three or more panels. FIG. 13 shows an exemplary product reservoir 300 having a first side panel 302, and opposing second side panel 304, and a bottom panel 306. Exemplary product reservoir 300 can be made by adding a gusseted bottom portion to the bag or pouch. Side gussets can alternatively or additionally be employed depending on a desired product reservoir size and shape. Other product reservoir design approaches can also be used in the aerosol products provided herein.

When filled, the reservoir can occupy no more than 80% of the height of the cavity at the longest point, alternatively no more than 70%, or no more than 60%. When filled, the reservoir can occupy more than 50% of the diameter of the cavity at the widest point, alternatively more than 60%, more than 70%, more than 80%, or more than 90%.

Product reservoirs constructed from sheets of material can include different material types and can be a single layer or multiple layers. In one form, the product reservoirs are typically 3-layer, or 4-layer pouches made respectively of polyacrylate/aluminum/polypropylene or polyethylene (PA/ALU/PP or PE) or polyethylene terephthalate/aluminum/orientated polyamide/polypropylene or polyethylene (PET/ALU/OPAi PP or PE). Although not limited to such, the product reservoirs can comprise about 20 grams to about 250 grams, about 30 grams to about 200 grams, or about 35 grams to about 100 grams, of a product composition, with the smaller capacity being useful as a trial product. Other product reservoir capacities are contemplated herein depending on the application of the aerosol product.

Aerosol products provided herein have improved ergonomics and functionality. Referring again to FIGS. 3 and 4, product container 102 has a height 122 and a width or effective/equivalent cross-sectional diameter 124. In one form, product container height 122 is no greater than 300 millimeters, 250 millimeters, 200 millimeters, or 175 millimeters, and product container width 124 is no greater than 75 millimeters, 66 millimeters, 53 millimeters, or 45 millimeters. Other heights and widths, however, can be used. The pressure container may have a volume from about 100 cubic centimeters to about 500 cubic centimeters, from about 100 cubic centimeters to about 400 centimeters, from about 150 cubic centimeters to about 300 cubic centimeters, or from about 170 cubic centimeters to about 230 cubic centimeters, for example.

Cavity 103 can have an empty volume that is not more than 10 times, not more than 8 times, or not more than 6 times a product capacity volume of product reservoir 130. In some forms, cavity 103 can have an empty volume that is from about 1 to about 10 times, about 2 to about 8 times, about 3 to about 7 times, or about 4 to about 6 times, a product capacity volume of product reservoir 130. “Empty volume” meaning an interior volume of container 102 in the absence of any structure or materials such as before assembly and adding a product composition, propellant, and adsorbent material.

In one form, the aerosol product can comprise a ratio of adsorbent material mass to product composition mass that is at least 0.55:1, at least 4:6, at least 1:1, at least 6:5, at least 7:5, at least 3:2, at least 7:4, or at least 2:1. In another form, the aerosol product the aerosol product can comprise a ratio of adsorbent material mass to product composition mass from about 3:5 to about 3:1, from about 4:6 to about 5:2, from about 1:1 to about 11:5, or from about 6:5 to about 2:1. In yet another form, the aerosol product can comprise a first ratio of empty cavity volume to product reservoir capacity volume, a second ratio of product composition mass within the product reservoir to adsorbent material mass, wherein the first ratio is greater than the second ratio.

Aerosol products provided herein can have a variety of spray properties. For example, in one form, the aerosol products have a total mass flow rate of the propellant/product composition mixture from about 0.05, 0.1, 0.15, or 0.2 grams/second to about 0.1, 0.15, 0.25, 0.3, 0.4, 0.6, or about 0.7 grams/second. The aerosol products can have, for example, a total mass flow rate of greater than or equal to 0.2 grams/second for greater than or equal to 50 seconds, greater than or equal to 60 seconds, greater than or equal to 70 seconds, greater than or equal to 80 seconds, greater than or equal to 90 seconds, greater than or equal to 100 seconds, greater than or equal to 110 seconds, or greater than or equal to 120 seconds. The spray rate can also be greater than 0.1 grams/second for greater than or equal to 100 seconds, greater than or equal to 110 seconds, greater than or equal to 120 seconds, greater than or equal to 130 seconds, greater than or equal to 140 seconds, greater than or equal to 150 seconds, greater than or equal to 160 seconds, or greater than or equal to 170 seconds. The amount of product composition delivered from a 2-second dispensing event can be from about 0.1, 0.2, or 0.3 grams to about 0.2, 0.5, 0.6, or 1 grams. The distance the product travels can be at least 6 inches (15.24 centimeters), at least 8 inches (20.32 centimeters), at least 10 inches (25.4 centimeters), at least 12 inches (30.48 centimeters), at least 14 inches (35.56 centimeters), at least 16 inches (40.64 centimeters), and at least 18 inches (45.72 centimeters). In one form, a surface area covered when dispensed at 18 inches (45.72 centimeters) can be from about 1 inches squared to about 8 inches squared, from about 2 inches squared to about 6 inches squared, or from about 2 inches squared to about 5 inches squared.

The aerosol products can deliver consistent consumer-acceptable aerosolized compositions, including the amount of product composition (without the propellant) from one use to the next throughout the life of the product. In addition to the amount of product delivered, the mean aerosol droplet size and distribution remain consistent from one spray to the next. The aerosol dispenser can dispense the product as a spray that includes a plurality of droplets having a distribution of droplet sizes, where the distribution can be quantified in terms of the size at which 50% of the distribution is below a particular size—this is commonly referred to as Dv50. The spray can include droplets having a Dv50 value from about 20 microns to about 140 microns, or from about 30 microns to about 60 microns, for example. Additionally, it can be advantageous to have a small fraction of large droplets to create an even coating on the targeted body part. As such, the Dv90, or size which 90% of a particular size, may be from about 60 microns to about 140 microns, or from about 60 microns to about 90 microns. To reduce inhalation risk, it can be beneficial for only a small percentage of droplets to be smaller than 10 microns; for example, less than 15%, 10%, or 5% of the total volume of dispensed droplets is under 10 microns. A Malvern Spraytec instrument can be used to evaluate aerosol droplet sizes. The Malvern Spraytec instrument uses a technique of laser diffraction for measurement of the size of the spray droplets. The intensity of light scattered as a laser beam passes through a spray is measured. This data is then analyzed to calculate the size of the droplets that created the scattering pattern. A Malvern Spraytec 2000 is used according to the manufacturer's instructions, with the test samples at a temperature between 20° C. to 22° C.

The aerosol products provided herein can be useful for dispensing product compositions related to personal care including, but not limited to, deodorants and antiperspirants, hairsprays, dry shampoos, and sunscreens, fabric care, air care (e.g., air fresheners), hard and soft surface care (e.g., oven cleaners, dusting sprays, furniture polishers), pesticides such as bug repellants and insecticides, herbicides, cooking sprays, and the like. Personal care compositions, such as deodorant and antiperspirant compositions, can be used for treating underarms or other areas of the body prone to sweating, including chest, underboob, legs, thighs, stomach, feet, arms, hands, and intimate areas.

Exemplary deodorant and antiperspirant compositions will now be described. All ingredient percentages described herein are by weight of the product composition, unless specifically stated otherwise, and may be designated as “wt %.” All ratios are weight ratios, unless specifically stated otherwise. All such percentages or weights as they pertain to listed ingredients are based on the active level and, therefore, do not include carriers or by-products that may be included in commercially available materials. The number of significant digits conveys neither a limitation on the indicated amounts nor on the accuracy of the measurements. Unless otherwise indicated, all measurements are understood to be made at approximately 25° C. and at ambient conditions, where “ambient conditions” means conditions under about 1 atmosphere of pressure and at about 50% relative humidity.

In some embodiments, it may be desirable for the viscosity of the deodorant or antiperspirant composition to be from about 1,000 centipoise, 2,000 centipoise, or 3,000 centipoise to about 50,000 centipoise 40,000 centipoise, or 30,000 centipoise, or 20,000 centipoise, or 10,000 centipoise, or 5,000 centipoise or 4,000 centipoise at 25° C. (1 centipoise being equal to 1×10⁻³ Pa·s). It is believed that a viscosity lower than 1,000 centipoise may lead to a deodorant or antiperspirant composition, which when spayed, results in a runny or drippy effect on skin. This may be perceived by a user as having a wet rather than dry feel.

In some embodiments, it may be desirable for the viscosity of the deodorant or antiperspirant composition to be from about 1 centipoise, 2 centipoise, or 3 centipoise to about 500 centipoise, 400 centipoise, 300 centipoise, 200 centipoise, 50 centipoise, 40 centipoise, or 10 centipoise at 25° C. This may aid in forming small aerosol droplets and/or fully evacuating the reservoir of the deodorant or antiperspirant composition.

The deodorant or antiperspirant compositions can comprise one or more non-volatile silicone fluids. The non-volatile silicone fluid may function as the primary or principal liquid carrier for the antiperspirant active. As used herein, the term “non-volatile” refers to a material that has a boiling point above 250° C. (at atmospheric pressure) and/or a vapor pressure below 0.1 mm Hg at 25° C. Conversely, the term “volatile” refers to a material that has a boiling point less than 250° C. (at atmospheric pressure) and/or a vapor pressure about 0.1 mm Hg at 25° C. The total concentration of non-volatile silicone fluids may be from about 30%, 35%, 40%, 45%, 50% to about 70%, 65%, 60%, 55% or 50% by weight of a deodorant or antiperspirant composition. In some embodiments, the total concentration of non-volatile, silicone fluids may be from about 35% to about 55% by weight of a deodorant or antiperspirant composition.

Some non-volatile, silicone fluids that may be used include, but are not limited to, polyalkyl siloxanes, polyalkylaryl siloxanes, and polyether siloxane copolymers, and mixtures thereof. Some preferred non-volatile silicone fluids may be linear polyalkyl siloxanes, especially polydimethyl siloxanes (e.g., dimethicone). These siloxanes are available, for example, from Momentive Performance Materials, Inc. (Ohio, USA) under the tradename Element 14 PDMS (viscosity oil). Silicones Fluids from Dow Corning Corporation (Midland, Mich., USA) available under the trade name Dow Corning 200 Fluid series (e.g., 3 to 350 centistokes). Other non-volatile silicone fluids that can be used include polymethylphenylsiloxanes. These siloxanes are available, for example, from the General Electric Company as SF 1075 methyl phenyl fluid or from Dow Corning as 556 Fluid. A polyether siloxane copolymer that may be used is, for example, a dimethyl polyoxyalkylene ether copolymer fluid. Such copolymers are available, for example, from the General Electric Company as SF-1066 organosilicone surfactant. The non-volatile silicone fluid may have an average viscosity from about 3 centistokes, 5 centistokes, 10 centistokes, 20 centistokes, or 50 centistokes to about 350 centistokes, 200 centistokes, 100 centistokes, 50 or 30 centistokes at 25° C. (1 centistoke being equal to 1×10⁻⁶ m²/s). In some specific embodiments, the silicone fluid may have a viscosity from about 5 centistokes to about 100 centistokes or 5 centistokes to about 50 centistokes or about 5 centistokes to about 30 centistokes. In some instances, the non-volatile silicone fluid is a polydimethylsiloxane fluid (also commonly referred to as dimethicone).

A deodorant or antiperspirant composition may also comprise one or more liquid fragrance materials. Liquid fragrance materials are typically a mixture of perfume or aromatic components that are optionally mixed with a suitable solvent, diluent or carrier. Some or many of the perfume components, when combined, may result in a highly polar liquid fragrance material. Some suitable solvents, diluents or carriers for the perfume components may include ethanol, isopropanol, diethylene glycol monoethyl ether, dipropylene glycol, diethyl phthalate, triethyl citrate, isopropyl myristate and mixtures thereof. A deodorant or antiperspirant composition may comprise from about 2%, 3% or 4% to about 10%, 8%, 6%, or 4% by weight of a liquid fragrance material.

The perfume component may be any natural or synthetic perfume component known to one skilled in the art of creating fragrances including, but not limited to, essential oils, citrus oils, absolutes, resinoids, resins, concretes, etc., and synthetic perfume components such as hydrocarbons, alcohols, aldehydes, ketones, ethers, acids, esters, acetals, ketals, nitriles, etc., including saturated and unsaturated compounds, aliphatic, carbocyclic and heterocyclic compounds. Some non-limiting examples of perfume components include: geraniol, geranyl acetate, linalool, linalyl acetate, tetrahydrolinalool, citronellol, citronellyl acetate, dihydromyrcenol, dihydromyrcenyl acetate, tetrahydromyrcenol, terpineol, terpinyl acetate, nopol, nopyl acetate, 2-phenylethanol, 2-phenylethyl acetate, benzyl alcohol, benzyl acetate, benzyl salicylate, benzyl benzoate, styrallyl acetate, amyl salicylate, dimethylbenzyl carbinol, trichloromethylphenyl-carbinyl acetate, p-tert.butyl-cyclohexyl acetate, isononyl acetate, vetiveryl acetate, vetiverol, alpha-n-amylcinammic aldehyde, alpha-hexylcinammic aldehyde, 2-methyl-3-(p-tert.butylphenyl)-propanol, 2-methyl-3-(p-isopropylphenyl)-propanal, 3-(p-tert.butylphenyl)-propanal, tricyclodecenyl acetate, tricyclodecenyl propionate, 4-(4-hydroxy-4-methylpentyl)-3-cyclohexene carbaldehyde, 4-(4-methyl-3-pentenyl)-3-cyclohexene carbaldehyde, 4-acetoxy-3-pentyltetrahydropyran, methyldihydrojasmonate, 2-n-heptylcyclopentanone, 3-methyl-2-pentylcyclopentanone, n-decanal, 9-decenol-1, phenoxyethyl isobutyrate, phenyl-acetaldehyde dimethyl acetal, phenylacetaldehyde diethyl acetal, geranonitrile, citronellonitrile, cedryl acetate, 3-isocamphylcyclohexanol, cedryl methyl ether, isolongifolanone, aubepine nitrile, aubepine, heliotropine, coumarin, eugenol, vanillin, diphenyl oxide, hydroxycitronellal, ionones, methylionones, isomethylionones, irones, cis-3-hexenol and esters thereof, indane musk fragrances, tetralin musk fragrances, isochroman musk fragrances, macrocyclic ketones, macrolactone musk fragrances, ethylene brassylate, and aromatic nitro-musk fragrances. Some perfume components are also described in Arctander, Perfume and Flavour Chemicals (Chemicals), Vol. I and II (1969) and Arctander, Perfume and Flavour Materials of Natural Origin (1960).

A deodorant or antiperspirant composition may comprise a variety of particulate materials. However, it is believed that the type (e.g., hydrophilic v. hydrophobic) and concentrations of particulate materials included in a deodorant or antiperspirant composition may, in some instances, impact skin feel, release of the antiperspirant active, and the propensity for clogging in the spray device. For example, too much antiperspirant active may result in a wet or sticky skin feel due to the propensity of antiperspirant actives to become sticky when hydrated (e.g., by perspiration). In addition, too much of a hydrophobic particulate material may reduce release of the antiperspirant active from the composition. Conversely, inclusion of a hydrophilic particulate material may advantageously aid release of the antiperspirant active, which may be beneficial in a composition comprising a high concentration of a non-volatile silicone fluid. However, hydrophilic materials may increase the risk of clogging in the presence of water. Therefore, it may be desirable to balance these and other design considerations when incorporating particulate materials in a deodorant or antiperspirant composition comprising a non-volatile silicone fluid that is in turn used in a spray device especially those with low propellant concentration.

It is believed that a deodorant or antiperspirant composition comprising a total non-volatile liquid material to total particulate material ratio (L/P ratio) from about 0.6, 0.8, 1, 1.2, or 1.4 to about 2.3, 2.2, 2.1, 2, 1.9, 1.8 or 1.6 may balance the tradeoff between enough particulates to provide acceptable skin feel while minimizing the appearance of residue. A deodorant or antiperspirant composition may have a total particulate concentration from about 30%, 35%, or 40% to about 60%, 55%, or 50% by weight of the antiperspirant composition, in keeping with the total liquid to total particulate (L/P) ratios previously described. While increasing the concentration of particulates may improve skin feel, it may also lead to an increased risk of clogging especially at low propellant concentrations.

Some examples of particulate materials include, but are not limited to, antiperspirant actives, powders (e.g., starch materials), encapsulated fragrance materials and bulking or suspending agents (e.g., clay materials, such as those described below). Other types of particulates may also be incorporated in a deodorant or antiperspirant composition.

A deodorant or antiperspirant composition can comprise one or more antiperspirant actives. The antiperspirant active may be any particle having antiperspirant activity. The antiperspirant active is preferably insoluble in the liquid components of the antiperspirant composition. Since the amount of antiperspirant active may significantly impact skin feel, a deodorant or antiperspirant composition may comprise from about 14% 16%, 18%, 20%, 22%, or 24% to about 38%, 36%, 34%, 32%, 30%, 28%, or 26% by weight of a particulate antiperspirant active. In some instances, it may be desirable to utilize a low concentration of the antiperspirant active, such as less than 20% or 18% by weight of the antiperspirant composition. The antiperspirant active concentrations refer to the anhydrous amount that is added. The antiperspirant active may represent the highest concentration of particulate materials in the antiperspirant composition. For example, the antiperspirant active (on an anhydrous basis) may comprise from about 50% to about 80%, or from about 50% to about 75%, or from about 55% to about 70% of the total particulate materials in the antiperspirant composition. The balance of the total particulate concentration comprises non-antiperspirant active particulates.

Some examples of suitable antiperspirant actives include astringent metallic salts, particularly including the inorganic and organic salts of aluminum. Some non-limiting examples exemplary aluminum salts that can be used include aluminum chloride and the aluminum hydroxyhalides having the general formula Al₂(OH)ₐQ_{b}XH₂O where Q is chloride, bromide, or iodide (preferably chloride), a is from about 2 to about 5, and a+b=about 6, and a and b do not need to be integers, and where X is from about 1 to about 6, and X does not need to be an integer. Particularly preferred are the aluminum chlorhydroxides referred to as “5/6 basic chlorhydroxide” wherein “a” is 5 and “2/3 basic chlorhydroxide” wherein “a” is 4. Aluminum salts of this type can be prepared in the manner described more fully in U.S. Pat. Nos. 3,887,692; 3,904,741; and 4,359,456. Preferred compounds include the 5/6 basic aluminum salts of the empirical formula Al₂(OH)₅DI2H₂O; mixtures of AICl₃6H₂O and Al₂(OH)₅Cl₂H₂O with aluminum chloride to aluminum hydroxychloride weight ratios of up to about 0.5.

The balance of the total particulate concentration of a deodorant or antiperspirant composition may comprise excipient particulate materials that are substantially inert with respect to the non-volatile silicone fluid. The excipient particulate materials may be either hydrophilic or hydrophobic (including hydrophobically modified, which tend to be moderately hydrophobic). Some non-limiting examples of substantially inert excipient particulate materials that may be included in a deodorant or antiperspirant composition include, but are not limited to, encapsulated fragrance materials; native starches such as tapioca, corn, oat, potato, and wheat starch particulates; talc; calcium carbonate; perlite; mica and polyethylene beads.

The substantially inert particulates may be free flowing. A deodorant or antiperspirant composition may comprise from about 0.25%, 0.5%, 1%, 5%, 10%, 12%, or 14% to about 25%, 22%, 20%, 18%, or 16% by weight of the antiperspirant composition of substantially inert particulates.

One substantially inert particulate material believed to be suitable for use is a hydrophilic or hydrophobically modified tapioca material. A tapioca material may be particularly beneficial as it is unlikely to induce an allergic reaction if inhaled. Tapioca is a starch which may be extracted from the cassava plant, typically from the root, which may then be processed or modified as known in the art. Tapioca starches are, advantageously, substantially non-allergenic. One non-limiting example of a hydrophobically modified tapioca material suitable for use comprises a silicone grafted tapioca starch, which is available under the trade name Dry Flo TS from AkzoNobel of the Netherlands. The INCI name is tapioca starch polymethylsilsesquioxane and may be produced by a reaction of methyl sodium siliconate (polymethylsilsesquioxane) and tapioca starch. This silicone grafted tapioca material is commercially available as CAS No. 68989-12-8. The silicone grafted tapioca material can be formed using any known means, including, but not limited to those methods described in U.S. Pat. Nos. 7,375,214, 7,799,909, 6,037,466, 2,852,404, 5,672,699, and 5,776,476. Other non-limiting examples of hydrophobically modified tapioca materials that are suitable for use include Dry Flo AF (silicone modified starch from Akzo Nobel), Rheoplus PC 541 (Siam Modified Starch), Acistar RT starch (available from Cargill) and Lorenz 325, Lorenz 326, and Lorenz 810 (available from Lorenz of Brazil). In some specific embodiments, the tapioca material may be hydrophilic to facilitate release of the antiperspirant active during use. One non-limiting example of a hydrophilic tapioca material suitable for use is available under the trade name Tapioca Pure available from Akzo Nobel. In some specific embodiments, the substantially inert particulate material comprises a hydrophilic tapioca material, a hydrophobic tapioca material or a mixture thereof.

A deodorant or antiperspirant composition may comprise one or more particulate fragrance carriers. Fragrance carriers are typically particulates, which would be considered part of the total particulate concentration of the antiperspirant composition. The fragrance carriers are preferably hydrophobic to minimize particle-to-particle interactions. The fragrance carriers may be either full or empty. A full fragrance carrier is a fragrance carrier that encapsulates or otherwise contains a perfume component while the fragrance carrier is stored within the spray device. Full fragrance carriers may release their perfume components by a variety of mechanisms post-delivery from the spray device to provide a desired aroma or fragrance experience for a user. For example, the perfume components may be released by moisture upon wetting of the fragrance carrier, e.g., by perspiration or other body fluids. Alternatively, or in addition thereto, the perfume components may be released by fracture of the carrier, such as by the application of pressure or a shearing force. An empty fragrance carrier is a fragrance carrier that does not contain a perfume component while stored within the spray device. One non-limiting example of an empty fragrance carrier is an uncomplexed cyclodextrin material.

Some non-limiting examples of fragrance carriers suitable for encapsulating a perfume component include, but are not limited to, oligosaccharides (e.g., cyclodextrins), starches, polyethylenes, polyamides, polystyrenes, polyisoprenes, polycarbonates, polyesters, polyacrylates, vinyl polymers, silicas, and aluminosilicates. Some examples of fragrance carriers are described in U.S. Patent Application Publication Nos. 2010/0104611; 2010/0104613; 2010/0104612; 2011/0269658; 2011/0269657; 2011/0268802; and U.S. Pat. Nos. 5,861,144; 5,711,941; 8,147,808; and 5,861,144.

A deodorant or antiperspirant composition may comprise from about 0.25%, 0.5%, 0.75%, 1%, or 2% to about 20%, 16%, 12%, 10%, 8%, 6% or 4% by weight of the composition of fragrance carriers. In some instances, the substantially inert excipient particles of the antiperspirant composition consist essentially of or completely of full fragrance carriers, empty fragrance carrier, or mixtures thereof. A deodorant or antiperspirant may comprise from about 0.25%, 0.5%, 0.75%, or 1% to about 6%, 4% or 2% by weight of the composition of full fragrance carriers. A deodorant or antiperspirant composition may comprise from about 0.25%, 0.5%, 1%, or 2% to about 16%, 12%, 10%, 8%, 6% or 4% by weight of the composition of empty fragrance carriers. In some instances, it may be desirable to incorporate a mixture of empty fragrance carriers and full fragrance carriers in the composition, wherein the empty fragrance carriers may be included to achieve the desired overall particulate concentration without the risk of perfume over-dosing.

In some instances, it may be desirable to provide a mixture of fragrance carriers and native starch powders to achieve the desired particle concentration. For example, from about a 20:80 to 80:20 (fragrance carrier to starch) mixture might be utilized. In some instances, a 50:50 mixture might be utilized and in other instances the native starch powders might have a concentration equal to about or less than 6% by weight of the deodorant or antiperspirant composition while the concentration of the fragrance carriers might be equal to about or less than 9% by weight of the composition.

A deodorant or antiperspirant composition may comprise a clay material as a bulking or suspending agent. The concentration of clay material may be from about 1%, 2%, 3% to about 8%, 6%, 5%, or 4% by weight of the antiperspirant composition. In some embodiments, the concentration of the clay material is from about 2% to about 6% by weight of the composition. In some embodiments, the total particulates of the composition may comprise from about 5% to about 20% or 5% to 15% of a clay material. In some embodiments clay materials are organoclays, which may be derived from clay minerals in which a portion of the inorganic cationic counter ions (e.g., sodium cations) of the clay mineral have been exchanged for organocations (e.g., quaternary ammonium chloride) thereby rendering the material organophilic rather than hydrophilic. Shearing/milling of the clay material deagglomerates the clay material platelets after which a polar clay activator may be added in some instances to further separate the platelets and promote the formation of hydrogen bonds between the edges of adjacent platelets. This enables formation of a higher volume three dimensional clay structure that suspends the particulates of the composition. This also increases the volume of the clay material in the composition, thereby increasing the volume or bulk of the total powder of the composition. This is also why the settling height of a deodorant or antiperspirant composition may be one quantitative/qualitative measure of the amount/quality of activation of a clay material.

Some non-limiting examples of clay materials include montmorillonite clays and hydrophobically treated montmorillonite clays. Montmorillonite clays are those which contain the mineral montmorillonite and may be characterized by having a suspending lattice. Some examples of these clays include but are not limited to bentonites, hectorites, and colloidal magnesium aluminum silicates. Some non-limiting examples of organoclays include modified bentonite, modified hectorite, modified montorlinite and combinations thereof, some examples of which are available under the trade names Bentone 27 (stearalkonium bentonite), Bentone 34 (stearalkonium bentonite) and Bentone 38 (disteardimonium hectorite) from Elementis Specialities Plc. and Tixogel VPV (quaternium 90-bentonite), Tixogel VZV (stearalkonium bentonite), Tixogel LGM (stearalkonium bentonite) and Claytone SO (stearalkonium bentonite) from Southern Clay Products. In some instances, the bulking and suspending material consists substantially of, essentially of and/or primarily of a clay material and more preferably an organoclay material. In these instances, the deodorant or antiperspirant composition may be substantially or completely free of silica materials used as a bulking/suspending material.

The deodorant or antiperspirant composition may also comprise a clay activator, such as propylene carbonate, triethyl citrate, methanol, ethanol, acetone, water and mixtures and derivatives thereof. Without intending to be bound by any theory, it is believed that the clay activator enhances the hydrogen bonds between the edges of adjacent clay platelets. Too little clay activator may provide insufficient hydrogen bonding between clay platelets while too much may create very strong interactions resulting in formation of agglomerates and loss of the desired bulking benefit. The clay activator may have a concentration ranging from 1:3 to 2:3 parts clay activator to clay material. Clay activators may also strongly interact with a deodorant or antiperspirant active (e.g., leading to clumping or coating of an antiperspirant active and/or changes in active polymer structure which may reduce antiperspirant efficacy). Therefore, it may be desirable to limit the amount of clay activator present in the composition to between about 0.5%, 0.75%, 1%, 1.25%, or 1.5% to about 3%, 2%, or 1.75% by weight of the composition.

It is believed that certain liquid materials may help maintain and/or promote the clay bulking and suspending benefit in a deodorant or antiperspirant composition that comprises a non-volatile silicone liquid, and optionally a liquid fragrance material, by facilitating increased interaction or loose bonding between the non-volatile silicone fluid and the clay material. A deodorant or antiperspirant composition can comprise at least one liquid activation enhancer. The at least one liquid activation enhancer, or the combination of a plurality of activation enhancers, may have a total concentration from about 2%, 4%, 6%, 8%, 10% to about 30%, 25%, 20%, 18%, 16%, 14%, 12%, 10% or 8% by weight of the antiperspirant composition. In some embodiments, the liquid activation enhancer has a concentration from about 2% to about 15% by weight of the antiperspirant composition. It is believed that concentrations higher than 30% may impact spreading of the antiperspirant composition on skin by increasing the surface tension of the composition, which is one mechanism by which a dry skin feel may be imparted in a deodorant or antiperspirant composition comprising a non-volatile silicone fluid.

Some non-limiting examples of liquid activation enhancers can include isopropyl myristate, isopropyl palmitate, ethyl stearate, methyl stearate, propyl stearate, butyl stearate, ethyl myristate, ethyl palmitate, butyl palmitate, propyl stearate, propyl palmitate, methyl stearamide, ethyl stearamide, isopropyl stearamide, ethyl palmitamide propyl palmitamide, stearyl benzoate, palmityl benzoate, C12-15 alkyl benzoate, benzyl palmitate, benzyl stearate, dodecylenbenezene and palmityl acetate. Liquid activation enhancers might also include fatty branched chain alcohols and ethoxylated fatty alcohols.

A deodorant or antiperspirant composition may comprise less than 30%, 20%, 10%, or less than 5% by weight of liquid materials other than non-volatile, silicone fluids. Said in another way, the liquid materials of the composition may comprise more than 70%, 75%, 80%, 85%, 90% or about 100% by weight of non-volatile silicone fluids.

It is believed that a deodorant or antiperspirant composition whose liquid materials comprise too much of a volatile silicone fluid may lead to an increased propensity for the appearance of a residue due to the evaporation of the volatile silicone fluid. A deodorant or antiperspirant composition may comprise less than 10%, 5%, 1%, or 0.5% by weight of a volatile silicone fluid. A deodorant or antiperspirant composition may be substantially or completely free of a volatile silicone fluid.

A deodorant or antiperspirant composition may comprise one or more silicone gums. A silicone gum may be added to a deodorant or antiperspirant composition to further increase substantivity of the antiperspirant composition and/or increase aerosol droplet size and/or increase deposition on the skin. However, formulating a deodorant or antiperspirant composition with a silicone gum in combination with relatively high concentrations of a non-volatile silicone fluid and/or relatively high concentrations of total particulates may involve a number of tradeoffs. For example, too much of a silicone gum may dramatically increase viscosity of the antiperspirant composition and the risk of clogging of the container actuator and/or valve, particularly where there is already a relatively high concentration of total particulates. Furthermore, too much of a silicone gum may reduce the diameter of the spray making it more difficult for a user to achieve complete coverage of an axillia (typically a 7.5 centimeters×12.5 centimeters area) during application as well as potentially creating regions of high antiperspirant composition dosage, thereby negatively impacting skin feel. Still further, the amount of gum required to control the deposition on skin and diameter of the spray pattern is dependent on the level and/or type of propellent, with the amount needed generally increasing as the propellant level and pressure increases.

Given the one or more potential challenges associated with incorporating a silicone gum, a deodorant or antiperspirant composition may be substantially or completely free of silicone gum materials. When inclusion of a silicone gum is desirable for antiperspirant products with less than about 70% propellant, a deodorant or antiperspirant composition may have a concentration from about 0.05% or 0.075% to about 0.75%, 0.5%, 0.4%, 0.3%, or 0.2% of a silicone gum by weight of the composition. When inclusion of a silicone gum is desirable for products with more than about 70% propellant, a deodorant or antiperspirant composition may have a concentration from about 0.05% or 0.1% to about 0.3%, 0.5%, 1.2%, 1.5%, or 2% of a silicone gum by weight of the composition. The silicone gum material may have a viscosity from about 100,000 centistokes to about 10,000,000 centistokes at 25° C.

If a silicone gum is included, any silicone gum having a viscosity within the ranges described herein may be used, provided it is soluble in the liquid carrier, propellant or a combination thereof of the deodorant or antiperspirant composition. Some suitable silicone gums include silicone polymers of the dimethyl polysiloxane type, which may have other groups attached, such as phenyl, vinyl, cyano, or acrylic, but the methyl groups should be in a major proportion. Silicone polymers having a viscosity below about 100,000 centistokes (molecular weight below about 100,000) at 25° C. are not considered silicone gums here but are rather, typically, considered a silicone fluid. One non-limiting example of silicone gum suitable for use is a silicone/gum fluid blend comprising a dimethiconol gum having a molecular weight form about 200,000 to 4,000,000 along with a silicone fluid carrier with a viscosity from about 0.65 to 100 mm² s⁻¹. An example of this silicone/gum blend is available from Dow Corning, Corp. of Michigan, USA under the trade name DC-1503 Fluid or XIAMETER® PMX-1503 FLUID (85% dimethicone fluid/15% dimethiconol). Other silicone gums materials include SF1236 Dimethicone, SF1276 Dimethicone, and CF1251 Dimethicone available from Momentive Performance Materials, Inc. of NY, USA.

A deodorant or antiperspirant composition is preferably substantially or completely free of water added as separate ingredient (i.e., anhydrous), as too much added water may result in several deleterious effects such as: 1) increasing the propensity for antiperspirant active particulates to agglomerate (thereby increasing the propensity for clogging), and 2) reducing dry feel on skin. It will be appreciated that even an anhydrous antiperspirant composition may still contain some water that is bound with an ingredient (e.g., antiperspirant active, starch, etc.) otherwise added to the antiperspirant composition. Additional examples of antiperspirant or deodorant compositions can be found in U.S. Patent Application Publication No. 2015/0023883, incorporated herein by reference.

### Exemplary Product Compositions

The following examples are given solely for the purpose of illustration and are not to be construed as limitations of the invention as many variations thereof are possible without departing from the spirit and the scope of the invention. Composition Examples 1-9 are homogenous single-phase solutions with Newtonian viscosities, and Composition Example 10 is an oil-in-water emulsion with a non-Newtonian viscosity. The composition examples can be made by any known method that creates single-phase or oil-in-water emulsions. Composition Examples 1-10 are intended for use as deodorant compositions that are typically applied topically to the underarms or other areas of the body prone to sweating including chest including underboob, legs including thighs, stomach, feet, arms, hands, and privates.

| | Ex. 1 | Ex. 2 | Ex. 3 | Ex. 4 | Ex. 5 | Ex. 6 |
|---|---|---|---|---|---|---|
| | (wt. %) | (wt. %) | (wt. %) | (wt. %) | (wt. %) | (wt. %) |
| Cyclopentasiloxane | Q.S. | Q.S. | Q.S. | Q.S. | Q.S. | Q.S. |
| Isopropyl Myristate | 10.00 | 10.00 | 10.00 | 0.00 | 0.00 | 0.00 |
| PPG-14 Butyl Ether | 0.00 | 0.00 | 0.00 | 10.00 | 10.00 | 10.00 |
| Ethanol | 0.00 | 20.00 | 30.00 | 0.00 | 20.00 | 30.00 |
| Piroctone Olamine | 0.10 | 0.10 | 0.10 | 0.10 | 0.10 | 0.10 |
| (commercially available as | | | | | | |
| Octopirox ™ from Clariant ®) | | | | | | |
| Triethyl Citrate | 1.00 | 1.00 | 1.00 | 1.00 | 1.00 | 1.00 |
| Fragrance | 2.00 | 2.00 | 2.00 | 2.00 | 2.00 | 2.00 |
| Citric Acid | 1.00 | 1.00 | 1.00 | 1.00 | 1.00 | 1.00 |

| | Ex. 7 | Ex. 8 | Ex. 9 | Ex. 10 |
|---|---|---|---|---|
| | (wt. %) | (wt. %) | (wt. %) | (wt. %) |
| Ethanol | Q.S. | | | 30 |
| Caprylic/Capric | 12.00 | | | 20 |
| Triglyceride | | | | |
| Dipropylene Glycol | 14.00 | 67.52 | 61.0 | |
| Propylene glycol | | 24.50 | | |
| Water | 5 | Q.S. | Q.S. | Q.S. |
| Citric Acid | | Ex | 1.0 | 0.35 |
| Triethylcitrate | | | 1.0 | 1 |
| Piroctone olamine | | 0.10 | 0.1 | 0.10 |
| Polysorbate 20 | | | | 0.30% |
| Sodium Magnesium | | | | 0.50% |
| Fluorosilicate | | | | |
| Xanthan Gum | | | | 0.05% |
| Fragrance | 1.50 | 2 | 2 | 2 |

### Exemplary Aerosol Products

A. An aerosol product, comprising:
   a. a container comprising a cavity and a top member;
   b. a dispensing assembly comprising:
      i. a valve;
      ii. an actuator for operating the valve;
      iii. a spray nozzle; and
      iv. a conduit for conveying a product to be dispensed from the container;
   c. a product reservoir disposed within the cavity and in fluid communication with the conduit;
   d. a product composition disposed within the product reservoir;
   e. a non-hydrocarbon propellant disposed within the cavity;
   f. adsorbent material disposed within the cavity; and
   g. a filter for mitigating escape of adsorbent material from the container.
B. The aerosol product of Paragraph A, wherein the filter is configured and positioned so as not to interfere with the product reservoir upon filling.
C. The aerosol product of Paragraph A, wherein the filter is configured and positioned so that it is not displaced upon filling the product reservoir
D. The aerosol product of Paragraph A, wherein the filter is positioned in one of an area within the cavity between a sealed top portion of the product reservoir and the top member, an area within the cavity below a sealed bottom portion of the product reservoir, and outside of the cavity.
E. The aerosol product of any one of the preceding paragraphs, wherein the product reservoir comprises from about 50 grams to about 250 grams, from about 30 grams to about 200 grams, or from about 35 grams to about 100 grams of the product composition.
F. The aerosol product of any one of the preceding paragraphs, wherein there is less than 7, 5, or 3 milliliters of residual product composition remaining within the product reservoir when the dispensing assembly will no longer dispense an aerosolized product composition.
G. The aerosol product of any one of the preceding paragraphs, wherein the product reservoir comprises at least two, or at least three panels.
H. The aerosol product of any one of the preceding paragraphs, wherein the product reservoir is a gusseted bag or pouch.
I. The aerosol product of Paragraph H, wherein the gusted bag or pouch comprises side gussets.
J. The aerosol product of Paragraph H, wherein the gusted bag or pouch comprises a bottom gusset.
K. The aerosol product of any one of the preceding paragraphs, wherein the container has a height that is no greater than 300 millimeters, no greater than 250 millimeters, no greater than 200 millimeters, or no greater than 175 millimeters, and a cross-sectional effective or equivalent diameter that is no greater than 75 millimeters, no greater than 66 millimeters, no greater than 53 millimeters, or no greater than 45 millimeters.
L. The aerosol product of any one of the preceding paragraphs, wherein the cavity comprises a volume that is not more than 10 times, not more than 8 times, or not more than 6 times a product capacity volume of the product reservoir.
M. The aerosol product of any one of the preceding paragraphs, wherein a ratio of adsorbent material mass to product composition mass is at least 0.55:1, at least 4:6, at least 1:1, at least 6:5, at least 7:5, at least 3:2, at least 7:4, or at least 2:1.
N. The aerosol product of any one of the preceding paragraphs, wherein a volume of the adsorbent material is greater than a volume of the product composition.
O. The aerosol product of any one of the preceding paragraphs, wherein a volume of the adsorbent material is at least 10% greater than, at least 20% greater than, or at least 30% greater than the volume of the product composition.
P. The aerosol product of any one of the preceding paragraphs, wherein the product comprises a first ratio of cavity volume to a product capacity volume of the product reservoir, wherein the product comprises a second ratio of mass of the product composition within the product reservoir to mass of the adsorbent material within the cavity, and wherein the first ratio is greater than the second ratio.
Q. The aerosol product of any one of the preceding paragraphs, wherein a first 2-second dispensing event comprises a mean droplet size, and wherein a twentieth 2-second dispensing even comprises a mean droplet size that is no more than 10% different from that of the first 2 second dispensing event
R. The aerosol product of any one of the preceding paragraphs, wherein a product composition mass dispensed for a 2-second dispensing event from the container varies less than 10% over the life of the aerosol product.
S. The aerosol product of any one of the preceding paragraphs, wherein the container comprises a fill pressure of from about 45 psi (310 kPa) to about 180 psi (1241 kPa), from about 60 psi (414 kPa) to about 125 psi (862 kPa), or from about 75 psi (517 kPa) to about 100 psi (689 kPa).
T. The aerosol product of any one of the preceding paragraphs, wherein the product composition comprises a deodorant composition that is substantially free or completely free of an aluminum-containing active.
U. The aerosol product of any one of the preceding paragraphs, wherein the product composition comprises an antiperspirant active.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be understood by those skilled in the art that various other changes and modifications can be made without departing from the spirit and scope of the present disclosure. It is therefore intended to cover in the appended claims all such changes and modifications that are within the scope of the present disclosure.

What is claimed is:

1. An aerosol product, comprising:
a. a container comprising a cavity and a top member;
b. a dispensing assembly in sealing engagement with the top member, the dispensing assembly comprising:
i. a valve;
ii. an actuator for operating the valve;
iii. a spray nozzle; and
iv. a conduit for conveying a product to be dispensed from the container;
c. a product reservoir disposed within the cavity and in fluid communication with the conduit;
d. a product composition disposed within the product reservoir;
e. a non-hydrocarbon propellant disposed within the cavity;
f. adsorbent material disposed within the cavity; and
g a filter for mitigating escape of adsorbent material from the container;
h, wherein the filter is configured and positioned so as not to interfere with the product reservoir upon filling.

2. The aerosol product of claim 1, wherein the cavity comprises a volume that is from about 1 to about 10 times a product capacity volume of the product reservoir.

3. The aerosol product of claim 1, wherein the container has a height that is no greater than 300 millimeters, and a cross-sectional diameter that is no greater than 75 millimeters.

4. The aerosol product of claim 1, wherein a volume of the adsorbent material is greater than a volume of the product composition.

5. The aerosol product of claim 1, wherein a ratio of adsorbent material mass to product composition mass is from about 3:5 to about 3:1.

6. The aerosol product of claim 1, wherein the product comprises a first ratio of cavity volume to a product capacity volume of the product reservoir;
wherein the product comprises a second ratio of mass of the product composition within the product reservoir to mass of the adsorbent material within the cavity; and
wherein the first ratio is greater than the second ratio.

7. The aerosol product of claim 1, wherein an aerosolized product composition dispensed from the container comprises a droplet distribution Dv90 value from about 60 microns to about 140 microns.

8. The aerosol product of claim 1, wherein the filter is positioned in an area within the cavity between a sealed top portion of the product reservoir and the top member.

9. The aerosol product of claim 1, wherein the filter is positioned in an area within the cavity below a sealed bottom portion of the product reservoir.

10. The aerosol product of claim 1, wherein the filter is positioned outside of the cavity.

11. The aerosol product of claim 1, wherein there is less than 7 milliliters of residual product composition remaining within the product reservoir when the dispensing assembly will no longer dispense an aerosolized product composition.

12. The aerosol product of claim 1, wherein the product reservoir comprises at least two panels.

13. The aerosol product of claim 1, wherein the product reservoir is a gusseted bag or pouch.

14. The aerosol product of claim 13, wherein the gusted bag or pouch comprises side gussets.

15. The aerosol product of claim 13, wherein the gusted bag or pouch comprises a bottom gusset.

16. The aerosol product of claim 1, wherein a product composition mass dispensed for a 2-second dispensing event from the container varies less than 10% over the life of the aerosol product.

17. The aerosol product of claim 1, wherein the container comprises a fill pressure of from about 45 psi to about 180 psi.

18. The aerosol product of claim 1, wherein the product composition comprises a deodorant composition.

19. The aerosol product of claim 18, wherein the deodorant composition is free of an aluminum-containing active.

20. The aerosol product of claim 18, wherein the deodorant composition comprises an aluminum-containing active.
